(19) United States
(12) Patent Application Publication
(10) Pub. No.: US 2023/0174284 A1
(43) Pub. Date: Jun. 8, 2023

(54) Anxiety Treatment Kit

(71) Applicant: Jessica Ibanez, Fredericksburg, VA (US)

(72) Inventor: Jessica Ibanez, Fredericksburg, VA (US)

(21) Appl. No.: 18/062,132

(22) Filed: Dec. 6, 2022

Related U.S. Application Data
(60) Provisional application No. 63/286,692, filed on Dec. 7, 2021.

(51) Int. Cl. B65D 77/08; A61K 36/185; A61J 1/03; A61K 47/55

(52) U.S. Cl. CPC B65D 77/08; A61K 36/185; A61J 1/03; A61K 47/551

(57) ABSTRACT

This present invention relates to an anxiety treatment kit for an individual to help reduce anxiety and panic in the individual. The anxiety treatment kit includes a plurality of anxiety coping items from which a user can choose to remain calm and grounded. The anxiety coping items include, but are not limited to, rubber bands, lavender oil, sour powder, CBD products, cold and squishy items, inspirational cards and meditation or relaxation tips to relax and calm a user during the time of an anxiety attack. The anxiety treatment kit may be made available both as a one-time purchase or in a subscription-based model. The box of the present invention is reusable, and features an easy open lid to provide convenient access to the anxiety coping items stored therein.

### CROSS-REFERENCE TO RELATED APPLICATION

The present application claims priority to, and the benefit of, U.S. Provisional Application No. 63/286,692, which was filed on Dec. 7, 2021 and is incorporated herein by reference in its entirety.

### FIELD OF THE INVENTION

The present invention relates generally to the field of anxiety reduction or treatment related products. More specifically, the present invention relates to a kit for aiding in addressing the anxiety of the user and comprises a box or other package filled with various coping items to help reduce or treat anxiety and or panic or at least diminish the effect of such issues. The coping items include rubber bands, oils, sour powder, squishy items, cold items, inspirational cards, meditation/relaxation tips, combinations thereof and more, which help the user suffering from anxiety or panic to remain calm and grounded, thereby preventing the users from hurting themselves, or causing disruption or inconvenience. Additionally, the anxiety kit of the present invention is lightweight and portable and can be used easily and conveniently. Accordingly, this disclosure makes specific reference thereto the present invention. Nonetheless, it is to be appreciated that aspects of the present invention are also equally applicable to other like applications, devices and methods of manufacture.

### BACKGROUND OF THE INVENTION

By way of background, anxiety disorders are one of the most common mental illness across the globe. According to the World Health Organization, 3.6 percent of the population, or about 264 million individuals worldwide, have some sort of anxiety disorder. In the U.S., anxiety disorders affect about 40 million adults in the United States age 18 and older, or roughly 18.1% of the population every year. Anxiety disorders involve excessive fear or anxiety, worry and stress due to social interactions, personal health, work or a particular phobia. There are a multitude of anxiety disorders such as panic disorder, generalized anxiety disorder, agoraphobia (the fear of places that might cause feelings of anxiety), specific phobia, social anxiety disorder, post-traumatic stress disorder (PTSD), obsessive-compulsive disorder, and separation anxiety disorder, which affects the ability of many people to function normally in daily life. Individuals suffering from such anxiety disorders may opt for different treatment solutions to address the symptoms of anxiety.

Some people may treat with a therapist who may help the individual to overcome and manage their anxiety or phobia. Usually, a person may have to attend multiple therapy sessions to reduce the symptoms of anxiety disorder. However, attending therapy sessions can be costly and not everyone can afford such expense. Additionally, therapy can take a long time to have a positive effect on an individual's anxiety, worry, fear, etc. Traditional therapy are not instantly effective on the people with anxiety disorders and may also require treatment with prescription medication potentially creating dependencies or addictions to the prescribed pharmaceuticals.

Additionally, people may visit health care providers who recommend psychiatric treatment in order to deal with the anxiety or other mental illness disorders. Costs associated with medical treatment, healthcare center co-pays, prescription fees and other medical expenses may be costly, and therefore not affordable for many individuals, thereby forcing many people's conditions to be left untreated, leaving people to deal with their issues on their own. Additionally, some people may experience extreme panic or anxiety, and end up hurting themselves or others and/or causing damage to property. Taking prescription medications each day can be both frustrating and difficult for many patients. Unnecessary stress associated with such treatment may lead to further medical complications and even in-patient hospitalizations. Unfortunately, many people suffering from anxiety and other mental illnesses such as panic attacks may lack the means and resources to cope up with their anxiety-related issues.

Therefore, there exists a long felt need in the art for an anxiety treatment product that helps an individual to cope with anxiety, stress, worry, phobias and other related issues, and that enables the individual to calm his or her symptoms through an accessible and cost effective means. Additionally, there is a long felt need in the art for an anxiety treatment product that is relatively inexpensive for the patient, and that may be easily replaced or replenished when needed. Moreover, there is a long felt need in the art for an anxiety treatment product that takes an immediate calming effect, and quickly diverts the individual's mind from negative thoughts. Furthermore, there is a long felt need in the art for an anxiety treatment product that does not result in the physical side effects associated with many types of prescription medication, and that is easily portable and accessible. Finally, there is a long felt need in the art for an anxiety treatment product that is relatively inexpensive to manufacture and that is both safe and easy to use.

The subject matter disclosed and claimed herein, in one embodiment thereof, comprises an anxiety treatment kit having an easy to open box with a hinged lid, a base portion having a space to accommodate a plurality of anxiety coping items including, without limitation, a number of rubber bands, a lavender or other essential oil bottle with a dropper or a diffuser, a sour powder bottle, a CBD product with a dropper, cold squishy or conformable items, inspirational cards with written supportive quotes, and meditation cards or cards containing various relaxation tips. One or more of the above-referenced items may be used by individuals to immediately relax and calm themselves during an anxiety or panic attack.

In this manner, the novel anxiety treatment kit of the present invention accomplishes all of the forgoing objectives, and provides a relatively safe, convenient and cost-effective solution to alleviate the symptoms associated with various anxiety disorders, and helps in reducing anxiety and panic among individuals. The anxiety kit of the present invention is also user friendly, inasmuch as it is far less expensive than other alternatives, is portable, and quickly and easily calms the stress, worry, or anxiety of a user without any side-effects or medical complications. Additionally, the anxiety treatment kit allows the individual to choose an appropriate coping object from its numerous available options to help cope with anxiety, such that the chosen product suits the individual user and addresses their particular needs to assist in minimizing anxiety.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an anxiety treatment kit having an easy-to-open box with a hinged lid. The box further comprises a base portion having space to accommodate a number of different anxiety coping items including, without limitation, a plurality of rubber bands, a lavender or essential oil bottle with a dropper or diffuser, a sour powder bottle, a CBD product with a dropper, a cold squishy item, a plurality of inspirational cards each containing a written inspirational quote and a plurality of cards having meditation or relaxation tips.

In a further embodiment of the present invention, an anxiety treatment kit comprises an easy-to-open box having a hinged lid, and a base portion having space to accommodate a number of anxiety-coping items including, without limitation, a plurality of rubber bands and a lavender or essential oil bottle containing an essential oil and a dropper or diffuser for dispensing the essential oil. The essential oils may include one or a combination of the following scents: lavender, rose, rosemary, cedarwood, sandalwood, bergamont, orange, chamomile, clary sage, neroli, peppermint, spearmint, tea tree, ylang ylang, etc. The kit may also include one or more of the following additional items, namely a citric acid powder bottle, CBD product with a dropper, foam or other deformable and/or squishy item, an inspirational card having an inspirational quote printed thereon and mindful meditation tip cards. The user can choose from the various coping items to relax during an anxiety and/or stress attack.

In yet a further embodiment of the present invention, a subscription based mental wellness or health kit is disclosed and comprises a plurality of anxiety and stress coping physical items, as well as topical and oral compositions. The physical items include one or more rubber bands, deformable and squishy items, inspirational cards and meditation or relaxation tips. The topical and/or oral compositions include one or more cannabis products, essential oils, sour powder, vitamins and the like. The health/wellness kit may be shipped to the user based on the subscription model selected.

In yet another embodiment of the present invention, a method of creating and delivering a mental wellness kit to a subscriber is described. The method to order a kit is comprised of subscribing to a kit fulfillment site, and then entering the subscriber's personal information and preferences for the kit. An interim kit is then created for the subscriber based on the information provided, wherein the kit contains a first group of items and a second group of items. The contents of the kit is confirmed, and then the order is filled. Finally, the kit is delivered to the subscriber using the personal information provided by the subscriber.

The anxiety treatment kit of the present invention provides users with a box kit filled with various coping items to help reduce anxiety and panic, and that allows an individual to select different items that will enable the individual to cope with his or her anxiety, thereby ensuring that each individual will be able to access an item that best helps him or her in a time of need. The anxiety treatment kit is easily portable, thereby ensuring that it is readily available during an anxiety or panic attack.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of an anxiety treatment kit of the present invention in accordance with the disclosed architecture, wherein the anxiety treatment kit comprises a plurality of anxiety-coping items;

FIG. 2 illustrates a flow chart showing one potential method of subscribing to a service for the creation and delivery of a personalized anxiety treatment kit of the present invention in accordance with the disclosed architecture;

FIG. 3 illustrates a perspective view of a user using one potential embodiment of a coping item from an anxiety treatment kit of the present invention in accordance with the disclosed architecture;

FIG. 4 illustrates a perspective view of various different shapes of the anxiety treatment kit of the present invention in accordance with the disclosed architecture; and

FIG. 5 illustrates a flow chart showing one alternative method of subscribing to a service for the delivery of a personalized anxiety treatment kit of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for an anxiety treatment kit that can be used by an individual to cope with anxiety, stress, worry, phobias and other related issues, and that enables the individual immediately and effectively calm his or her symptoms. Additionally, there is a long felt need in the art for an anxiety treatment kit that is relatively inexpensive for the patient, and that may be easily replaced or replenished when necessary. Moreover, there is a long felt need in the art for an anxiety treatment kit that takes an immediate calming effect, and quickly diverts the individual's mind from negative thoughts. Furthermore, there is a long felt need in the art for an anxiety treatment kit that does not result in the physical side effects associated with many types of prescription medication, and that is easily portable and accessible. Finally, there is a long felt need in the art for an anxiety treatment kit that is relatively inexpensive to manufacture and that is both safe and easy to use.

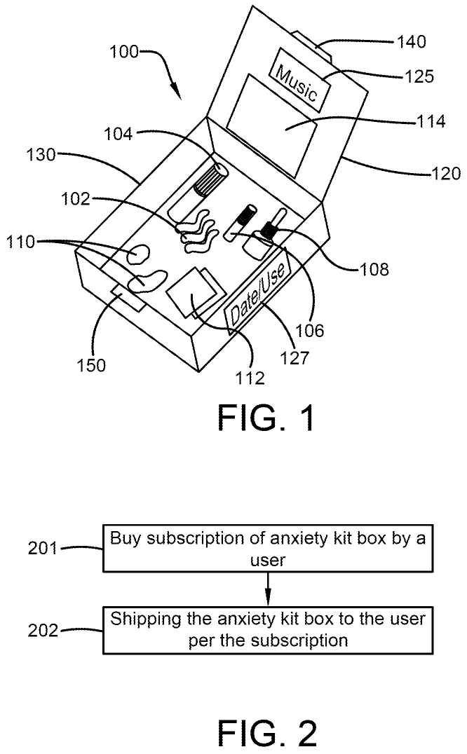

Referring initially to the drawings, FIG. 1 illustrates a perspective view of one potential embodiment of an anxiety treatment kit 100 of the present invention in accordance with the disclosed architecture, wherein the anxiety treatment kit 100 comprises a plurality of anxiety-coping items, including one or more physical items and topical or oral items. As shown, the anxiety kit box 100 has an easy-to-open lid 120 and contains a collection of anxiety-coping physical items, such as written reminders, and easy activities that keep an individual grounded during an anxiety or panic attack, or other mental health related episode. The anxiety treatment kit 100 can have slots to keep the anxiety coping items secured within a pre-determined position within the box during transportation or use. While it is contemplated that many of the anxiety coping items described herein will be commercially available items, it is also contemplated that the kit 100 may also include prescribed items provided by a health professional, nutritionist or other provider of medical care related services.

In one embodiment, a generally rectangular or other quadrate configuration is provided with dimensions of the anxiety kit box 100 of approximately 12.75(L)×9.5(W)×3.75(H) inches. Alternatively, the dimensions may be 10(L)×8(W)×2.5(H) inches, or any other suitable shape or size that satisfies user need and/or preference and that is easily portable. The anxiety kit box 100 is preferably both sturdy and durable, and can be made out of premium quality board, wood and paper. The anxiety kit box 100 is an elegant-looking, reusable, easy-to-open and eco-friendly box. The anxiety kit box 100 comes with a lid 120, a base 130 having space to accommodate various anxiety coping items of different shapes and size, a magnetic, VELCRO® or manual fastener 140 which is attached to the male/female part 150, and an anti-microbial coating 160 to prevent the transmission of germs, bacteria, viruses, infectious diseases and other harmful pathogens.

In the preferred embodiment, the anxiety kit box 100 includes different groups of products, such as a first group of physical items such as, but not limited to, rubber bands 102, squishy items or toys 110, inspirational cards 112, and meditation or relaxation tips 114 to relax and calm a user during the time of an anxiety attack. The kit 100 may further include a second group of topical or oral items, such as, but not limited to, lavender or essential oils 104, sour powder 106, CBD products 108, vitamins, prescriptions, lotions and the like. As stated above, each anxiety coping item may have a slot on the base of the box 100 so that the items remain sorted and organized during transportation of the kit 100. The anxiety-coping items in the box 100 may be selected and/or personalized to alleviate anxiety, stress, panic and fatigue in a particular individual. In a further embodiment of the present invention, the kit 100 may also comprise a music box 125 which plays a song, tune or other soothing sounds such as an inspirational message when the lid 120 is opened. In addition, a usage counter 127 may be integrated into the box to keep track of, for example, how many times the lid 120 is opened for purposes of reporting the same to a therapist or other mental healthcare worker.

The CBD product 108 preferably contains less than 0.3 percent THC, and is below the legal limit for pesticides, heavy metals and molds. Further, the CBD product 108 is preferably made from U.S.-grown hemp and contains zero artificial colors, flavors and/or preservatives. The

CBD product helps to alleviate an individual's chronic pain, insomnia and anxiety. In one embodiment, the CBD product 108 is presented as a 30 mL bottle having a 1 mL dropper for dispensing the CBD product 108.

The lavender oil 104 is an essential oil that both promotes and supports relaxation. More specifically, lavender aromatherapy calms anxiety by impacting the limbic system. In the present invention, lavender oil 104 can be inhaled via a diffuser or steam. The lavender oil 104 of the present invention preferably contains pure lavender essential oil, and does not contain artificial fragrances. In one embodiment, the lavender oil 104 may be made available as a roll on, or may be presented in 5 mL, 10 mL or 20 mL bottles with a dropper. Nonetheless, the present invention is not limited to lavender oil, and other essential oils may also be provided for treatment of different maladies including, without limitation, rosemary, cedarwood, sandalwood, bergamont, orange, chamomile, clary sage, neroli, peppermint, spearmint, tea tree, ylang ylang and combinations thereof.

The sour powder 106 can be in the form of citric acid and helps to decrease oxidative stress. Vitamin C also helps calm a user, and places the user in a better and more relaxed frame of mind. In one embodiment, the sour powder 106 may be present in the form of a relatively small or miniature bottle that can be easily opened to remove the powder 106. The sour powder 106 may come in 10 gm, 20 gm or 50 gm bottles, or any other size bottle that satisfies user need and/or preference.

The squishy or deformable items or toys 110 are perfect stress relief items and are both safe and non-toxic. The squishy items 110 can be used as stress relievers or fidget toys, and are both comfortable and soft to touch. The items 110 can also be used for wrist, hand and finger coordination exercises, and are effective as ADHD/ADD autism toys. In the preferred embodiment, the squishy or deformable items or toys 110 are made of a material that is inherently cold (or that can be made cold by refrigerating the same) and gives a cool sensation to the user squeezing or otherwise handling the same. The relatively cold nature of the squishy item or toy 110 increases endorphins in the brain, which can ease symptoms of depression and anxiety and also decrease cortisol, a stress-inducing hormone. Additionally, the rubber bands 102 can be used by a user to alleviate stress. More specifically, the rubber bands 102 can be placed on the wrist and lightly snapped to distract the individual from other unpleasant thoughts.

A plurality of inspirational cards 112 with motivational and inspiring quotes printed thereon may also be included in the kit 100 to inspire the user. The cards 112 preferably have heartfelt, supportive and comforting messages to reduce the anxiety and stress of the user. Meditation or relaxation tips 114 are also present in the anxiety kit 100 to provide a user with meditation instructions, such as mindful meditation, heartful meditation and gratitude to help the user perform meditation.

FIG. 2 illustrates a flow chart showing one potential method of subscribing to a service for the creation and delivery of a personalized anxiety treatment kit 100 of the present invention in accordance with the disclosed architecture. As shown, a user can buy a monthly or quarterly subscription of the anxiety treatment kit 100 using a toll-free number or through an online portal using a smartphone application/website at Block 201. Then, at Block 202, the user enters personal information such as, but not limited to, the user's name, address, description of the conditions or symptoms being experienced, description of objects that soothe or provide comfort to the user, etc. At, Block 203, the user supplied information and data is used to create a personalized anxiety treatment kit 100 for the user. Finally, the personalized anxiety kit 100 is shipped to the user at Block 204. Along with the anxiety kit 100, self-help books and gratitude journals can also be shipped.

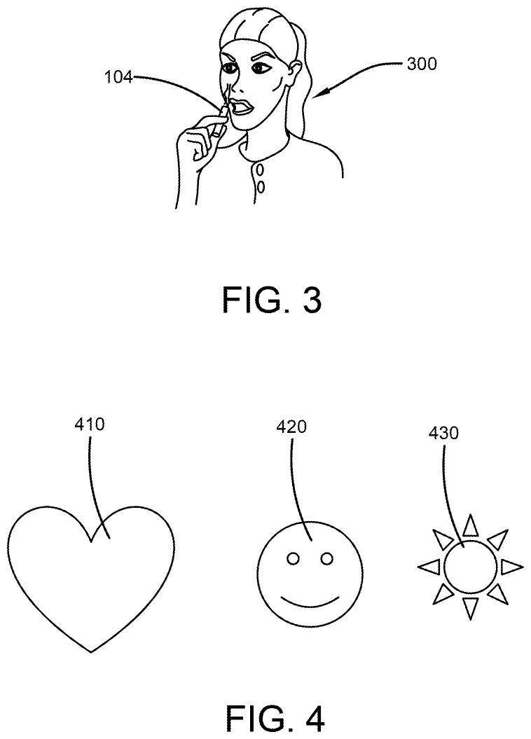

FIG. 3 illustrates a perspective view of a user 300 using one potential embodiment of a coping item from an anxiety treatment kit 100 of the present invention in accordance with the disclosed architecture. More specifically, the user is inhaling lavender oil 104 via a diffuser. The lavender oil 104 is a preferred component of the anxiety kit 100, and is the best essential oil for alleviating anxiety and stress. Nonetheless, other essential oils may also be selected depending on the requirements and type of anxiety an individual may be suffering from.

Additionally, the box containing the various coping items can be of any shape and size but is preferably rectangular, square or circular with a magnetic, zipper, hook and loop or VELCRO® fastener. The box may further comprise one or more logos, indicia, trademarks, geometric patterns, customizable colors and fonts, embroidery and prints and/or images on its outer or inner surfaces. FIG. 4 illustrates a perspective view of various different shapes of the anxiety treatment kit 100 of the present invention in accordance with the disclosed architecture. For example, a heart shaped box 410, a smiley faced box 420 and a star shaped 430, wherein the subscriber may select from these or other groups of design options.

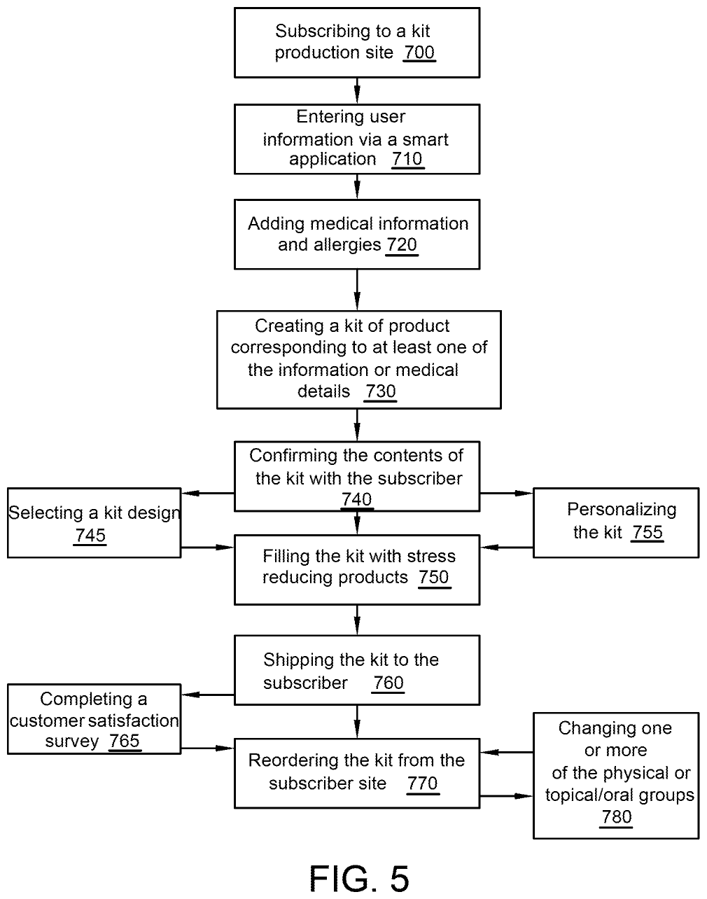

FIG. 5 illustrates a flow chart showing one alternative method of subscribing to a service for the delivery of a personalized anxiety treatment kit of the present invention in accordance with the disclosed architecture. The method begins with the user 300 initially subscribing to a kit fulfillment website at step 700, and then entering subscriber information at step 710 (similar to that described above). Next, the user may enter medical information 720, such as the type of anxiety from which they suffer as well as allergies and other information to prevent a reaction that may contemplate the treatment. The interim contents for the kit 100 are then created at step 730, and the subscriber confirms the contents of the kit 100 at step 740. The subscriber may select a shape of the box or package at step 745, and may also create other personalization at step 755. The personalization may include various indicia, information, graphics, coverings, tactile or other sensory queues. Once approved, the kit is then filled at step 750 and is shipped or otherwise delivered to the subscriber at step 760. The subscriber may be asked to complete a customer satisfaction survey at step 765. In addition, at step 770, the subscriber may reorder the kit 100 once the components have been consumer or otherwise finished. During the ordering process, the subscriber may change one or more of the physical group or topical/oral groups at step 780.

Certain terms are used throughout the following description and claims to refer to particular features or components. As one skilled in the art will appreciate, different individuals may refer to the same feature or component by different names. This document does not intend to distinguish between components or features that differ in name but not structure or function. As used herein “anxiety kit”, “anxiety box kit”, “anxiety relieving kit”, and “subscription based mental wellness kit”, are interchangeable and refer to the anxiety treatment kit 100 of the present invention.

Notwithstanding the forgoing, the anxiety treatment kit 100 of the present invention and its various components can be of any suitable size and configuration as is known in the art without affecting the overall concept of the invention, provided that it accomplishes the above stated objectives. One of ordinary skill in the art will appreciate that the size, configuration and components of the anxiety treatment kit 100 and its various components as shown in the FIGS. are for illustrative purposes only, and that many other sizes and shapes of the anxiety treatment kit 100 and its components are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An anxiety treatment kit comprising:
a box having a lid, the lid being hingedly attached to the box and openable to reveal an internal space;
a first group of items;
a second group of items distinct from the first group of items;
the first and second group of items disposed within the internal space of the box; and
the first and second group of items usable in the treatment of a mental condition.

2. The anxiety treatment kit as recited in claim 1, wherein the first group of items are a plurality of physical items.

3. The anxiety treatment kit as recited in claim 2, wherein the second group of items are a plurality of topical or oral items.

4. The anxiety treatment kit as recited in claim 3, wherein the plurality of physical items include one or more of a rubber band, a cold deformable item, a squishy item, an inspirational card, and a meditation or relaxation tip printed on an object.

5. The anxiety treatment kit as recited in claim 4, wherein the plurality of topical or oral items include at least one of a cannabis product, an essential oil, a sour powder, and a vitamin or a supplement.

6. The anxiety treatment kit as recited in claim 5, wherein the lid includes a music box for playing a song, a tune, a sound or an inspirational message.

7. The anxiety treatment kit as recited in claim 6 further comprising a date information and a counter for counting a frequency that the lid to the box has been opened.

8. The anxiety treatment kit as recited in claim 7, wherein the box has a shape selected from a circle, a square, a rectangular, a heart, a smiley face, or a sun shape.

9. The anxiety treatment kit as recited in claim 8, wherein the essential oil is comprised of at least one of a lavender, a rose, a rosemary, a cedarwood, a sandalwood, a bergamont, an orange, a chamomile, a clary sage, a neroli, a peppermint, a spearmint, a tea tree, and a ylang ylang oil.

10. The anxiety treatment kit as recited in claim 5, wherein the cannabis product is a CDB oil.

11. The anxiety treatment kit as recited in claim 1, wherein the anxiety treatment kit is personalized based on a user's information.

12. The anxiety treatment kit as recited in claim 11, wherein the user's information includes at least one of a personal, a medical and an allergy information.

13. A method of creating and delivering an anxiety treatment kit comprising the steps of;
subscribing to a kit fulfillment site by a subscriber;
entering subscriber information at the kit fulfillment site;
creating an interim kit for the subscriber based on the subscriber information, the interim kit containing a first group of items and a second group of items;
confirming the interim kit;
filling the interim kit to create the anxiety treatment kit; and
delivering the anxiety treatment kit to the subscriber.

14. The method as recited in claim 13, including a further step of selecting a shape of a box or package for the anxiety treatment kit prior to the step of delivering the anxiety treatment kit.

15. The method as recited in claim 14, including a further step of personalizing the anxiety treatment kit with at least one of an indicia, a graphic, an information, a covering, a tactile or other sensory que prior to the step of delivering the anxiety treatment kit.

16. The method as recited in claim 15, including a further step of completing a subscriber satisfaction survey after the step of delivering the anxiety treatment kit.

17. The method as recited in claim 16, including a further step of reordering the anxiety treatment kit after the step of delivering the anxiety treatment kit.

18. The method as recited in claim 17, including a further step of changing at least one of the first group or second group of items after the step of reordering the anxiety treatment kit.

19. An anxiety treatment kit comprising:
a package having a lid, the lid movable about a hinge and the package having an inner area; and
a group of physical items and a group of topical or oral items disposed within the inner area of the package, wherein each of the groups of physical items and topical or oral items being secured within the inner area, and further wherein the group of topical or oral items are selected based on user information.

20. The anxiety treatment kit as recited in claim 19, wherein the group of physical items include one or more of a rubber band, a cold deformable item, a squishy item, an inspirational card and a meditation or relaxation tip, and the group of topical or oral items include at least one of a cannabis product, an essential oil, a sour powder, and a vitamin.
